(19) United States
(12) Patent Application Publication
(10) Pub. No.: US 2019/0371437 A1
(43) Pub. Date: Dec. 5, 2019

(54) METHOD AND SYSTEM FOR PREDICTING CHANGE IN FUNCTIONAL PROPERTY OF BIOMOLECULE

(71) Applicant: Samsung Electronics Co., Ltd., Suwon-si (KR)

(72) Inventors: Priyadarshini Panemangalore PAI, Bangalore (IN); Rajasekhara reddy Duvvuru MUNI, Bangalore (IN); Garima AGARWAL, Bangalore (IN); Tae Yong KIM, Daejeon (KR)

(21) Appl. No.: 16/430,473

(22) Filed: Jun. 4, 2019

(30) Foreign Application Priority Data
Jun. 4, 2018 (IN) 201841020862
Apr. 17, 2019 (KR) 10-2019-0045143

(51) Int. Cl. G16C 20/50; C12N 9/10; G16C 20/30; G16C 10/00; G16C 20/20; G16C 20/60; G16C 20/70; G16C 20/80; G16C 60/00

(52) U.S. Cl. CPC G16C 20/50; C12N 9/1048; G16C 20/30; G16C 10/00; G16C 60/00; G16C 20/60; G16C 20/70; G16C 20/80; G16C 20/20

(57) ABSTRACT

A method for predicting a change in a functional property of a biomolecule includes obtaining a plurality of biomolecules and mutation data associated with at least one functional property; determining a net change in the at least one functional property based on at least one parameter associated with the mutation data for each biomolecule at a site; encoding a plurality of sequenced features for the mutation data associated with each biomolecule; configuring a prediction model to detect a relationship of the sequence features and the net change in the at least one functional property of the mutation data associated with each biomolecule; and predicting a change in the at least one functional property of a target biomolecule based on the prediction model for the mutation data at a user specified site.

### CROSS-REFERENCE TO RELATED APPLICATION

This application claims the benefits of Indian Patent Application No. 201841020862, filed on Jun. 4, 2018, in the Indian Intellectual Property Office, and Korean Patent Application No. 10-2019-0045143, filed on Apr. 17, 2019, in the Korean Intellectual Property Office, the disclosure of each is incorporated herein in its entirety by reference.

### BACKGROUND

### 1. Field

The present disclosure relates to enzyme engineering, and more specifically, it is related to a method and system for predicting change in a functional property of a biomolecule.

### 2. Description of the Related Art

Enzymes have diverse roles in biochemical processes sustaining life and they have the ability to accelerate reactions to several folds, which has been utilized for a wide range of applications in various industries. Gaining insights into how enzymes work has also enabled their biotechnological manipulation for resulting in greater product yield or ability to use a variety of substrates, etc. In order to manipulate enzymes for engineering of their functional properties (such as, enzyme-ligand affinity which signifies the strength of enzyme-ligand interaction), certain key residues are targeted in their architecture (which is made of a chain of amino acids referred to as the enzyme sequence, folded to form a three dimensional (3D) structure). These key residues have been naturally endowed with certain evolutionary and physicochemical properties that are different from other residues in the enzymes. Changing of these residues or sites can lead to changes in the enzyme architecture and eventually also, in the enzyme function.

Conventionally, the roles of these key residues are determined by mutational studies including substitutions (replacing the naturally occurring also known as wildtype residue with another residue), relevant residue insertions or deletions in the sequence. Such experimental studies for determining the roles of all the positions in the enzyme sequence with alternate amino acids substitutions, is cumbersome. This is because at every position along the length of the enzyme sequence (sometimes greater than few hundreds of amino acids), the observed amino acid (or the wildtype) can be substituted with for example, 19 other naturally occurring amino acids. Therefore, computerized predictive models are required for rapid assessment of the substitutions at these sites, reducing the search space for the evaluation of functional changes and effectively utilizing available resources to also select the substitution sites.

There are existing methods to predict sites for functional property engineering. But, there are following technical challenges associated with their use.

Lack of availability of input information (i.e., the number of enzyme-ligand interactions known are much more than the number of structure/complexes available. Therefore, these methods have limitations in usage),

Quantitative but specific application (i.e., the available methods target ligand binding regions in the enzyme and are not applicable to residues which may be functionally relevant and outside the pocket), and

Specific to enzymes (i.e., available methods are not suitable for handling the vast range of enzymes available as they have been developed on an enzyme or a group of them).

Thus, it is desired to address the above mentioned disadvantages or other shortcomings or at least provide a useful alternative.

### BRIEF DESCRIPTION OF THE DRAWINGS

These and/or other aspects will become apparent and more readily appreciated from the following description of the exemplary embodiments, taken in conjunction with the accompanying drawings in which:

This method is illustrated in the accompanying drawings, throughout which like reference letters indicate corresponding parts in the various figures. The embodiments herein will be better understood from the following description with reference to the drawings, in which:

FIG. 1 is a block diagram of an electronic device for predicting a change in the functional property of a biomolecule using mutation data, according to an embodiment as disclosed herein;

FIG. 2 is a flow diagram illustrating a method for predicting the change in the functional property of the biomolecule using the mutation data, according to an embodiment as disclosed herein;

FIG. 3 is a schematic overview of a system for predicting the change in the functional property of the biomolecule using the mutation data, according to the embodiment as disclosed herein;

FIG. 4 is an example scenario in which a dataset preparation is explained, according to the embodiment as disclosed herein;

FIG. 5 is an example scenario in which factors influencing enzyme function are explained, according to the embodiment as disclosed herein;

FIG. 6 is an example scenario in which a recursive feature elimination is performed using a linear model function to determine essential features for prediction, according to the embodiment as disclosed herein;

FIG. 7A is a graph illustrating different feature combinations for neighborhood sequence context, target site evolutionary changes, target site physicochemical changes and target site group-wise changes, according to the embodiment as disclosed herein;

FIG. 7B is a graph illustrating the importance of sequence features in the Random Forest (RF) model, according to the embodiment as disclosed herein;

FIG. 7C is a graph illustrating selection of number of neighbors for the prediction model development, according to the embodiment as disclosed herein;

FIG. 7D is a graph illustrating the performance of the prediction models during the training phase, according to the embodiment as disclosed herein;

FIG. 7E is a graph illustrating the performance of the prediction models upon large-scale validation on an independent test set, according to the embodiment as disclosed herein; and

FIG. 7F is a graph illustrating the performance of the best prediction model across different enzyme classes in the independent test set, according to the embodiment as disclosed herein.

### OBJECT OF THE EMBODIMENTS

The principal object of the embodiments herein is to provide a method and system for predicting change in a functional property of a biomolecule using mutation data.

Another object of the embodiment herein is to obtain a plurality of biomolecules and the mutation data associated with at least one functional property.

Another object of the embodiment herein is to determine a net change in a functional property based on at least one parameter (i.e., the kinetic parameter) associated with the mutation data for each biomolecule.

Another object of the embodiment herein is to encode a plurality of sequence features for the mutation data associated with each biomolecule.

Another object of the embodiment herein is to configure a prediction model to detect a relationship of the sequence features and the changes in the functional property of the mutation data associated with each biomolecule.

Another object of the embodiment herein is to identify sites in the biomolecule which provides desired changes in the functional property according to a user-defined and user-desired criteria.

Another object of the embodiment herein is to reduce a search space (for site selection and substitution selection) by using the prediction model, while predicting the change in the functional property of the biomolecule using the mutation data.

### SUMMARY

Accordingly embodiments herein disclose a method for predicting a change in a functional property of a biomolecule. The method includes obtaining a plurality of biomolecules and mutation data associated with at least one functional property. Further, the method includes determining a net change in a functional property based on at least one parameter associated with the mutation data for each biomolecule at a site. Further, the method includes encoding a plurality of sequence features for the mutation data associated with each biomolecule. Further, the method includes configuring a prediction model to detect a relationship of the sequence features and the change in the functional property of the mutation data associated with each biomolecule. Further, the method includes predicting a change in the functional property of a target biomolecule based on the prediction model, for the mutation data at a user-specified site. Further, the method can include mutagenizing the target biomolecule at the user-specified site to obtain a selected change in the at least one functional property of the target biomolecule based on the predicted change for a mutant residue at the user-specified site.

In an embodiment, the plurality of biomolecules and the mutation data associated with at least one functional property is obtained by obtaining at least one amino acid sequence for each biomolecule, mapping the mutation data on the sequence information for each biomolecule, mapping the parameter associated with the functional property on the mutation data, and generating a dataset and obtaining homology information for each biomolecule based on protein domain knowledge.

In an embodiment, the mutation data comprises a substitution site in the biomolecule sequence, a wildtype residue associated with the biomolecule at that site, one or more mutant residues associated with the biomolecule at that site, or a combination thereof.

In an embodiment, the net change in the functional property of the biomolecule associated with a substitution site is computed as the difference between the starting point value associated with the parameter (for example, a kinetic parameter) for the mutant and the starting point value for the wildtype residue for the same parameter, for a ligand and a set of assay parameters.

The plurality of the sequence features for the data associated with a biomolecule is encoded by a combination or sub-combination of the properties of the wildtype residue, properties of the mutant residue and their differences upon mutation at the target site and the target site neighborhood.

In an embodiment, the combination or sub-combination of the properties encoded for the target site is obtained by selecting at least one of a set of physicochemical and evolutionary properties of the target site and for the target site neighborhood by selecting factors comprising at least one of a polarity index, a secondary structure factor, a molecular size, a relative amino acid composition and an electrostatic charge for capturing the sequence context.

In an embodiment, the selected set of physicochemical and evolutionary properties for the wildtype and the mutant residue at the target site and the target neighborhood are encoded using a combination of a numeric component and a categorical component.

In an embodiment, the sequence context is encoded as an average of the factors computed over the wildtype and mutant neighborhood of the target site.

In an embodiment, the numeric component of the sequence features comprises a physicochemical property, wherein the physicochemical property comprises residue flexibility, residue volume, side-chain angle, radius of gyration, side-chain volume, molecular weight, polarity, relative frequencies in alpha-helix, relative frequencies in beta-sheet, relative frequencies in reverse-turn, Chou-Fasman parameter of coil formation, steric parameter, localized electric effect, or a combination thereof.

In an embodiment, the categorical component encodes the wildtype to mutant change in the amino acid physicochemical groups using one or more properties, wherein the one or more properties comprises size, aliphaticity, aromaticity, polarity and charge.

In an embodiment, the evolutionary property is determined using historically recorded data and the biomolecule homologs.

In an embodiment, the evolutionary property is indicative of site specific residue information transfer.

In an embodiment, the prediction model is configured by grouping the mutation data associated with each biomolecule into two groups based on a pre-defined grouping criteria applied to the net change in the at least one functional property, partitioning the mutation data associated with each biomolecule into a training dataset and a testing dataset based on a pre-defined partitioning criteria, presenting sequence features extracted from the biomolecule in the training set for detecting one or more partitioning functions describing the relationship between the sequence features and the net change, selecting a best partitioning function based on a pre-defined assessment criteria, and assessing the classification function performance of the selected partitioning function on the testing dataset.

In an embodiment, the pre-defined grouping criteria for grouping the mutation data associated with the biomolecule is based on one of increase and decrease in the net change of the at least one functional property.

In an embodiment, the at least one functional property comprises affinity of the target biomolecule towards a ligand.

In an embodiment, the target biomolecule comprises an enzyme.

In an embodiment, the prediction model is used to reduce the number of amino acids to be evaluated by mutagenesis of the target biomolecule at the user-specified site for a selected change in the at least one functional property.

In an embodiment, the prediction model is used to identify at least one site in the target biomolecule which provides a selected change in the at least one functional property.

Accordingly, embodiments herein disclose an electronic device for predicting a change in a functional property of a biomolecule. The electronic device includes a data extraction unit, a sequence feature detector, a prediction model generator and a functional property change analyzer. The data extraction unit is configured to obtain a plurality of biomolecules and mutation data associated with at least one functional property. The sequence feature detector is configured to encode a plurality of sequence features for the mutation data associated with each biomolecule. The prediction model generator is configured to configure a prediction model to detect a relationship of the sequence features and the net change in the at least one functional property of the mutation data associated with each biomolecule. The functional property change analyzer is configured to predict the change in the at least one functional property of the target biomolecule based on the generated prediction model, for the mutation data at one or more user specified sites. Optionally, the device further comprises a communicator configured to communicate internally between internal hardware components and with external devices and/or a user.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION

The embodiments herein and the various features and advantageous details thereof are explained at a greater depth with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks which carry out a described function or functions. These blocks, which may be referred to herein as units or modules or the like, are physically implemented by analog or digital circuits such as logic gates, integrated circuits, microprocessors, microcontrollers, memory circuits, passive electronic components, active electronic components, optical components, hardwired circuits, or the like, and may optionally be driven by firmware and software. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the invention. Likewise, the blocks of the embodiments may be physically combined into more complex blocks without departing from the scope of the invention

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

As used herein, the term “and/or” includes any and all combinations of one or more of the associated listed items. Expressions such as “at least one of,” when preceding a list of elements, modify the entire list of elements and do not modify the individual elements of the list. The terminology used herein is for the purpose of describing particular embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein. It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Accordingly, embodiments herein achieve a method for predicting change in a functional property of a target biomolecule. The method includes obtaining a plurality of biomolecules and mutation data associated with at least one functional property. Further, the method includes determining a net change in a functional property based on at least one parameter (for example, a kinetic parameter) associated with the mutation data for each biomolecule. Further, the method includes encoding a plurality of sequence features for the mutation data associated with each biomolecule. Further, the method includes configuring a prediction model to detect a relationship of the sequence features and the changes in the functional property of the mutation data associated with each biomolecule. Further, the method includes predicting the change in the functional property of each biomolecule based on the prediction model for the mutation data at a user-specified site. The method can further include mutagenizing the target biomolecule at the user-specified site to obtain a selected change in the at least one functional property of the target biomolecule based on the predicted change for a mutant residue at the user-specified site.

Unlike conventional methods and conventional systems, the proposed method can be used to predict changes in properties, for example, enzyme-ligand affinity changes upon single amino acid substitutions, based on sequence derived features using machine learning algorithms in a rapid and accurate manner.

The proposed method for predicting the directionality of change in the enzyme-ligand affinity, utilizes substitution site properties and associated sequence context capturing physicochemical and evolutionary information of the enzyme sites in association with the functional property.

In the proposed method, a prediction model generator utilizes specifically curated mutation datasets for rapid qualitative estimation of enzyme-ligand affinity changes upon single amino acid substitution using the sequence information. The method utilizes the relevant enzyme information for developing a predictive model and helps identify candidates for mutagenesis. The method can include mutagenizing the target biomolecule at the user-specified site to obtain a selected change in the at least one functional property of the target biomolecule based on the predicted change for a mutant residue at the user-specified site. The method utilizes sequence information, which is abundantly available. It is not limited to the ligand-binding region. And it can efficiently handle all classes of biochemically diverse enzymes (i.e., all six enzyme classes, namely oxidoreductase, transferase, hydrolase, ligase, isomerase and lyase) allowing wider applicability.

The proposed method can be used in synthetic biochemistry for enzyme engineering.

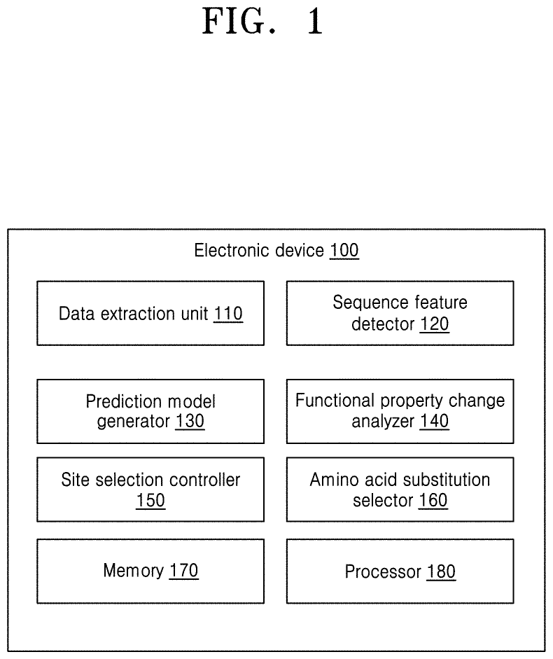

Referring now to the drawings, and more particularly to FIGS. 1 through 7F, there are shown exemplary embodiments.

FIG. 1 is a block diagram of an electronic device 100 for predicting change in a functional property of a biomolecule using mutation data, according to an embodiment as disclosed herein. The electronic device 100 can be, for example, but not limited to a cellular phone, a smart phone, a Personal Digital Assistant (PDA), a tablet computer, a laptop computer or the like. The biomolecule can be an enzyme.

In an embodiment, the electronic device 100 includes a data extraction unit 110, a sequence feature detector 120, a prediction model generator 130, a functional property change analyzer 140, a site selection controller 150, an amino acid substitution selector 160, a memory 170 and a processor 180.

In an embodiment, the data extraction unit 110 is configured to obtain a plurality of biomolecules and the mutation data associated with at least one functional property. In an embodiment, the functional property includes affinity of each biomolecule towards a ligand. In an embodiment, a range of values for the parameter, such as a kinetic parameter, associated with the functional property are obtained and mapped on the mutation data. In an embodiment, the mutation data for a biomolecule in the plurality of biomolecules comprises at least one of a substitution site associated with the biomolecule, a wildtype residue associated with the biomolecule at that site and one or more mutant residues associated with the biomolecule at that site. In an embodiment, a net change in the functional property based on a parameter (for example, a kinetic parameter) is computed as a difference of the starting point value of one of the mutant types and the starting point value of the wildtype, with respect to a site, for a ligand and a set of assay parameters. The net change is functionally relevant for the biomolecule. In an embodiment, all the sites for all the biomolecules mapped with a net change in the functional property based on the parameter form the dataset for predictive modeling.

Further, the sequence feature detector 120 is configured to encode a plurality of sequence features for the mutation data associated with each biomolecule. In an embodiment, the plurality of the sequence features for the data associated with each biomolecule is encoded by a combination or sub-combination of the properties of the wildtype residue, properties of the mutant residue and their differences upon mutation at a target site and the target site neighborhood, the target site neighborhood including the target site and at least one or more, but not more than three residues on either side of the target site.

In an embodiment, the combination or sub-combination of the properties encoded for the target site is obtained by selecting at least one of a set of physicochemical and evolutionary properties for the target site, and target site neighborhood by selecting factors comprising at least one of a polarity index, a secondary structure factor, a molecular size, a relative amino acid composition and an electrostatic charge for capturing the sequence context.

In an embodiment, the selected set of physicochemical and evolutionary properties for the wildtype and the mutant residue at the target site and the target neighborhood are encoded using combination of a numeric component and a categorical component.

In an embodiment, the sequence context is encoded as an average of the factors computed over the wildtype and mutant neighborhood of the target site.

In an embodiment, the numeric component of the sequence features comprises a physicochemical property, wherein the physicochemical property comprises residue flexibility, residue volume, side-chain angle, radius of gyration, side-chain volume, molecular weight, polarity, relative frequencies in alpha-helix, relative frequencies in beta-sheet, relative frequencies in reverse-turn, Chou-Fasman parameter of coil formation, steric parameter, localized electric effect, or a combination thereof.

In an embodiment, the categorical component encodes a wildtype residue to mutant residue change in amino acid physicochemical group using one or more properties, wherein the one or more properties comprises size, aliphaticity, aromaticity, polarity and charge.

In an embodiment, the evolutionary property is determined using historically recorded data and the biomolecule homologs. Further, the evolutionary property is indicative of site specific residue information transfer.

Further, the prediction model generator 130 configures a prediction model to detect a relationship of the sequence features and the change in the functional property of the mutation data associated with each biomolecule. In an embodiment, the prediction model is configured by grouping the mutation data associated with the biomolecule into two groups based on a pre-defined criteria applied on net functional property change, partitioning the mutation data associated with the biomolecule into a training dataset and a testing dataset based on a pre-defined criteria, presenting sequence features extracted from the biomolecule in the training set for detecting one or more partitioning functions capable of explaining the relation between the features and the net property change, selecting a best partitioning function based on pre-defined assessment criteria, and assessing the classification function performance on the testing dataset.

In an embodiment, the pre-defined grouping criteria for grouping the mutation data associated with the biomolecule is based on one of increase and decrease in the net change of the functional property.

Further, the functional property change analyzer 140 predicts the change in the functional property of a target biomolecule based on the prediction model, for the mutation data at a user specified site.

In an embodiment, the prediction model is used to reduce the number of amino acids to be evaluated at the user-specified site for desired changes in the functional property. Evaluation can be, for example, by mutagenizing the target biomolecule at the user-specified site, with or without subsequent functional testing. Further, the prediction model is used to identify at least one site in the biomolecule which provides desired changes in the functional property according to user-defined and user-desired criteria. In an embodiment, the site selection controller 150 provides a set of sites based on user-defined criteria for prediction of the change in the functional property. In an embodiment, the amino acid substitution selector 160 provides a set of substitutions based on the evaluation of the changes in the functional property at the site.

The processor 180 is configured to execute instructions stored in the memory 170 and to perform various processes. A communicator (not shown) is configured for communicating internally between internal hardware components and with external devices and/or a user via one or more networks.

The memory 170 stores instructions to be executed by the processor 180. The memory 170 may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory 170 may, in some examples, be considered a non-transitory storage medium. The term “non-transitory” may indicate that the storage medium is not embodied in a carrier wave or a propagated signal. However, the term “non-transitory” should not be interpreted that the memory 170 is non-movable. In some examples, the memory 170 can be configured to store larger amounts of information than the memory. In certain examples, a non-transitory storage medium may store data that can, over time, change (e.g., in Random Access Memory (RAM) or cache).

Although the FIG. 1 shows various hardware components of the electronic device 100 but it is to be understood that other embodiments are not limited thereon. In other embodiments, the electronic device 100 may include less or more number of components. Further, the labels or names of the components are used only for illustrative purpose and does not limit the scope of the invention. One or more components can be combined together to perform same or substantially similar function to predict the change in the functional property of the biomolecule using the mutation data in the electronic device 100.

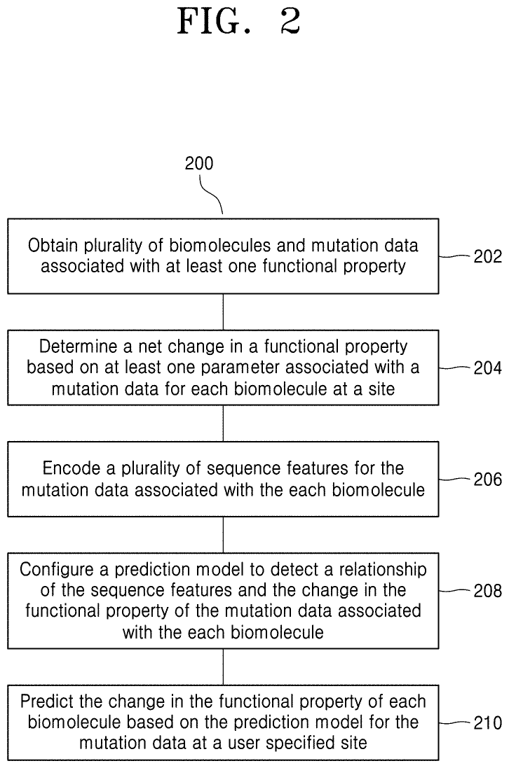

FIG. 2 is a flow diagram 200 illustrating a method for predicting the change in the functional property of the biomolecule using the mutation data, according to the embodiment as disclosed herein.

At 202, the method includes obtaining the plurality of biomolecules and mutation data associated with the one or more functional property. In an embodiment, the method allows the data extraction unit 110 to obtain the plurality of biomolecules and the mutation data associated with the one or more functional property.

At 204, the method includes determining the net change in the functional property based on at least one of parameter (i.e., desired kinetic parameter) associated with the mutation data for each biomolecule. In an embodiment, the method allows the data extraction unit 110 to determine the net change in the functional property based on at least one of parameter associated with the mutation data for each biomolecule.

At 206, the method includes encoding the plurality of sequence features for the mutation data associated with a site in each biomolecule. In an embodiment, the method allows the sequence feature detector 120 to encode the plurality of sequence features for the mutation data associated with each biomolecule.

At 208, the method includes configuring the prediction model to detect the relationship of the sequence features and the changes in the functional property in the mutation data associated with each biomolecule. In an embodiment, the method allows the prediction model generator 130 to configure the prediction model to detect the relationship of the sequence features and the changes in the functional property of the mutation data associated with each biomolecule.

At 210, the method includes predicting the change in the functional property of each biomolecule based on the prediction model, for the mutation data at the user specified site. In an embodiment, the method allows the functional property change analyzer 140 to predict the change in the functional property of each biomolecule based on the prediction model, for the mutation data at the user specified site.

The proposed method can be used to predict the enzyme-ligand affinity changes in a qualitative approach manner. Essentially, if an enzyme is given (sequence information) and the substitution is performed, the method can predict whether the effect on affinity is desirable or not. It is developed using supervised machine learning, which is a technique of inferring a function from labelled training data.

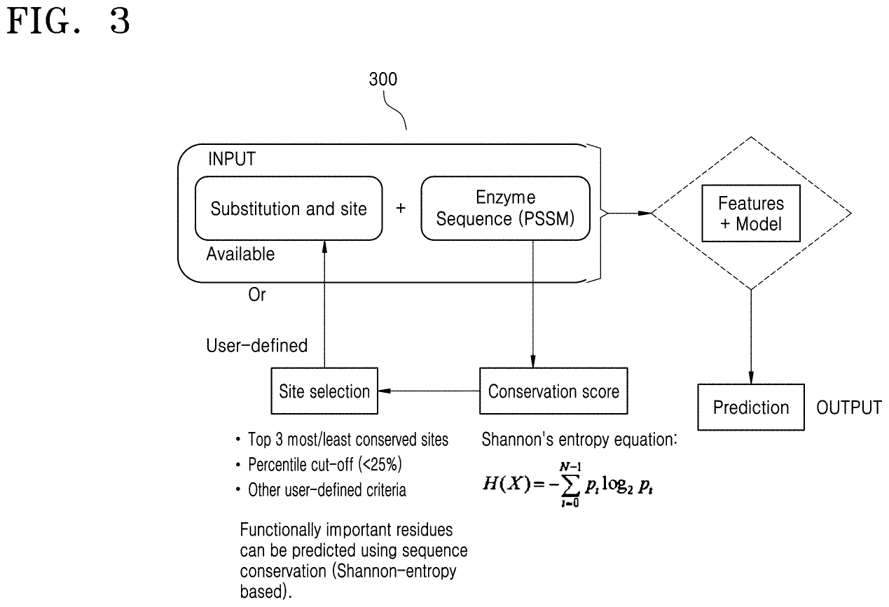

FIG. 3 is a schematic overview of a system 300 for predicting the change in the functional property of the biomolecule using the mutation data, according to the embodiment as disclosed herein.

For any supervised machine learning, the first requirement is to obtain the data associated with the biomolecule from various sources: In order to have data for the machine to learn from, in the proposed method, the electronic device 100 collects the enzyme data from various databases (e.g., UniProtKB or the like). For each of the enzymes, the electronic device 100 performs a processing step to obtain filtered and validated datapoints (X: amino acid substitutions). The processing step includes the following for obtaining the dataset with many datapoints (X and Y):

Enzymes with multiple UniProtKB identification (IDs) are excluded,

In a given enzyme, amino acid substitutions (wildtype-mutant pair) are recorded as datapoints,

All the datapoints mapped with a ligand were taken,

All the datapoints were mapped to affinity for the wildtype and the mutant type for the ligand and for the same type of experimental assays, and

Computation of affinity change and labelling of the datapoint (Y) as positive class (if change is negative, affinity has increased, desirable) and negative class (if change is positive, affinity has decreased, undesirable).

Unlike existing methods, the affinity change is not readily available for all substitutions, so that the electronic device 100 first obtains the records of wildtype and mutant type amino acids and their affinity parameters. Then, the affinity change (K_{M}) was computed as follows.

At a given position T in the sequence, the affinity parameter starting point value recorded for the wildtype is subtracted from the affinity parameter starting point value recorded for the mutant and assigned as the change in K_{M}. This forms the output variable (Y).

ΔK_{M}=K_{M}(i)Mutant−K_{M}(i)Wildtype

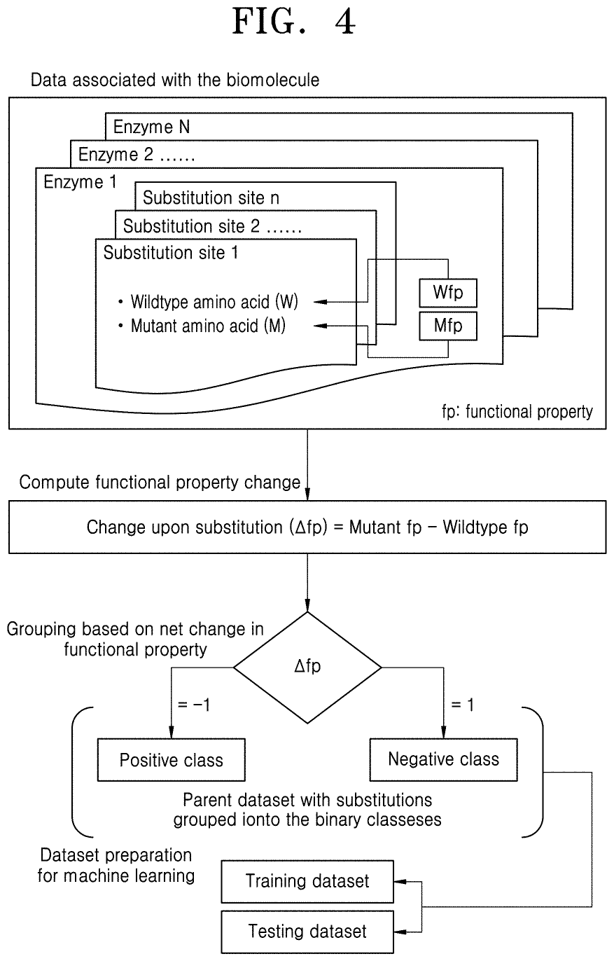

In an example, as shown in the FIG. 4, in order to assist the machine learning process from the available data, the electronic device 100 collects experimental records of enzymes with mutation sites and associated kinetic parameters (such as K_{M} which signifies enzyme-ligand affinity). Further, for each site in these enzymes, the electronic device 100 calculates the change in K_{M} to perform class labeling. If the sign of the above change is negative (−1), it is associated with increase in affinity and is desirable (Positive class). On the contrary, if the sign of the change is positive (+1), it is associated with a decrease in affinity and is not desirable (Negative class). The class labeling is done for every datapoint to form a benchmark dataset.

In a preferred embodiment, the prediction model generator 130 groups the mutation data associated with the biomolecule into two groups based on the pre-defined criteria applied on the net functional property change. Based on the pre-defined criteria, the prediction model generator 130 partitions the mutation data associated with the biomolecule into the training dataset and the testing dataset;

Further, the prediction model generator 130 presents the sequence features, extracted from the biomolecule in the training set for detecting one or more partitioning functions capable of explaining the relation between the features and the net property change.

Furthermore, the prediction model generator 130 presents selection of the best partitioning function based on the pre-defined assessment criteria and assesses the classification function performance on the testing dataset.

Further, the electronic device 100 ensures that there is representation from all the enzyme classes for handling biochemical diversity.

Sequence feature extraction: The sequence feature extraction is basically representing every datapoint with features or properties characteristic of the task under consideration. For the amino acid substitutions, the physicochemical properties, evolutionary and sequence neighborhood properties are extracted.

Below are the factors influencing enzyme function:

Changes involved at target site, and

Sequence neighborhood

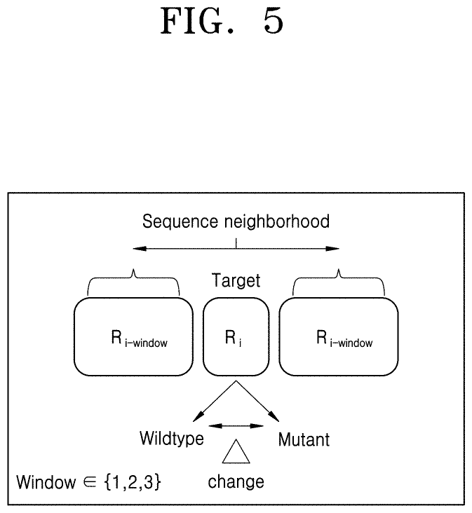

The physicochemical and evolutionary properties for the target site are explained below as shown in the FIG. 5:

Physicochemical Properties

Amino acid index features—Target position

1. wildtype value (wAAₙ) for R_{w}

2, mutant value (mAAₙ) for Rₘ

3. change upon substitution (dAAₙ)=mAAₙ−wAAₙ

AAₙ, where n∈{1-13}

Further, the numeric component of the sequence features are selected from a collection of the physicochemical properties are below:

Residue flexibility,

Residue volume,

Side chain angle,

Radius of gyration,

Side chain volume,

Molecular weight,

Polarity,

Relative frequencies in alpha-helix,

Relative frequencies in beta-sheet,

Relative frequencies in reverse-turn,

Chou-Fasman parameter of coil formation,

Steric parameter, and

Localised electric effect.

Therefore, the features related to the physicochemical properties are 13*3=39 features.

Further, the categorical component represents the transitions encoding in the amino acid physicochemical groups with one or more properties, where the properties are below:

Size ^{∈}{S_S, S_L, L_S, L_L},

Aliphaticity ^{∈}{Ali_Ali, Ali_Nali, Nali_Ali, Nali_Nali},

Aromaticity ^{∈}{Aro_Aro, Aro_Naro, Naro_Aro, Naro_Naro},

Polarity ^{∈}{P_P, P_Np, Np_P, Np_Np}, and

Charge ^{∈}{C_C, C_U, U_C, U_U}.

Group:

Size: Small (S) Large (L),

Aliphaticity: Aliphatic (Ali) Non-aliphatic (Nali),

Aromaticity: Aromatic (Aro) Non-aromatic (Naro),

Polarity: Polar (P) Non-polar (Np), and

Charge: Charged (C) Uncharged (U).

Therefore, the features related to the change in the amino acid groups at the target sites are 5*4=20 Features.

Evolutionary Properties

wildtype to mutant (AAy), and

Sign (AAy) for w→m.

where y∈PAM {40, 120, 250} and BLOSUM {45, 62, 90}

Therefore, the features related to the mutation matrices at the target site are 6*2=12 features.

wildtype to mutant (WOPw→m), and

Sign (WOPw→m).

where sign E{−1, 0, 1}; allowance criteria: if sign=−1, then substitution is not allowed; +1, then substitution is allowed as observed among Weighted Observed Percentages (WOP) in sequence homologs. Since, the score and allowance were found to be correlated, they have been used in a mutually exclusive manner while predictive modeling.

Therefore, the features related to the evolutionary features at the target site are 2*1=2 features.

Sequence Context

1. Wildtype residue neighborhood feature

(wFx)=Σ_{i-window}^{i+window}Factor_x_{R}_{_{w}}

2. Mutant residue neighborhood features

(mFx)=Σ_{i-window}^{i+window}Factor_x_{R}_{ₘ}

change upon substitution (dFx)=wFx−mFx

Factor_x, where x∈PROMAX [I, II, III, IV, V]

PROMAX Factors I-V:

Factor I: Polarity Index,

Factor II: Secondary structure factor,

Factor III: Molecular size or volume,

Factor IV: Relative amino acid Composition, and

Factor V: Electrostatic charge.

Therefore, the features related to the sequence context are 5*3=15

Features.

In an example, for all the enzymes, the electronic device 100 explores the substitution properties at the sequence level. Based on the existing methods, enzyme function depends on the key residues as well as their neighborhood. In the proposed method, the electronic device 100 encodes both of them and captures a unique combination of target site properties (i.e., 4 target site properties), involved physicochemical and evolutionary changes and associated sequence context.

The first target site property is based on native properties contributing to the enzyme architecture at the target position/site. For this, in an example, 13 out of 544 indices available in the AAindex are selected, the combination of which has not been used before. For each property, the electronic device 100 encodes the wildtype, mutant and associated change giving rise to 39 features.

The second target site property is based on whether this change at the target position is recorded as accepted in evolution. For this, in an example, the electronic device 100 uses the Point Accepted Mutation (PAM) and BLOcks

SUbstitution Matrix (BLOSUM) mutation matrices. For each matrix, the electronic device 100 not only encodes how often this change was seen (denoted by the probability score which is routinely used in similar studies) and but also, if at all it was acceptable (denoted by the score sign) giving rise to 12 features.

The third target site property is based on whether this change is similar or leads to a paradigm shift in nature of the amino acids at the site. For this, in an example, considered 5 different groups the size, aliphaticity, aromaticity, polarity and charge are considered, and changes encoded include, for example for size, Small to Small, Small to Large, Large to small and Large to large. This is also unique in the sense of encoding, giving rise to 20 features.

The fourth target site property is based on whether the substitution is favored evolutionarily. For encoding this information, sequence homologs of the query enzyme are collected and a weighted observed percentage of the desired wildtype to mutant change is encoded.

Besides these features of the target site, various physicochemical features of the target neighborhood are also considered and included. For this, the electronic device 100 indicates the averaged physicochemical properties over the wildtype neighborhood, mutant neighborhood and captured the associated change using PROMAX factors I-V derived from 54 selected physicochemical properties listed in the AAlndex. This gave rise to 15 features.

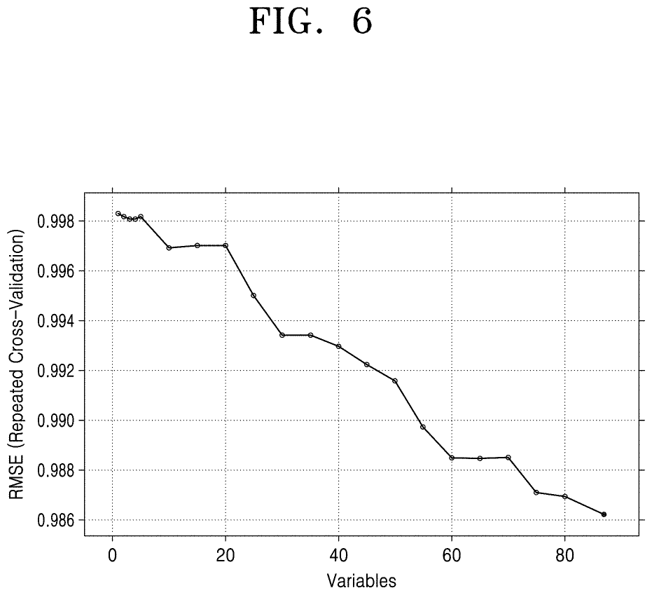

As shown in the FIG. 6, in order to test whether all the extracted features are needed, recursive feature elimination using a linear model function is performed. Results suggested that when all the features are used, the error in prediction is least, so that, using all these features, model development is achieved.

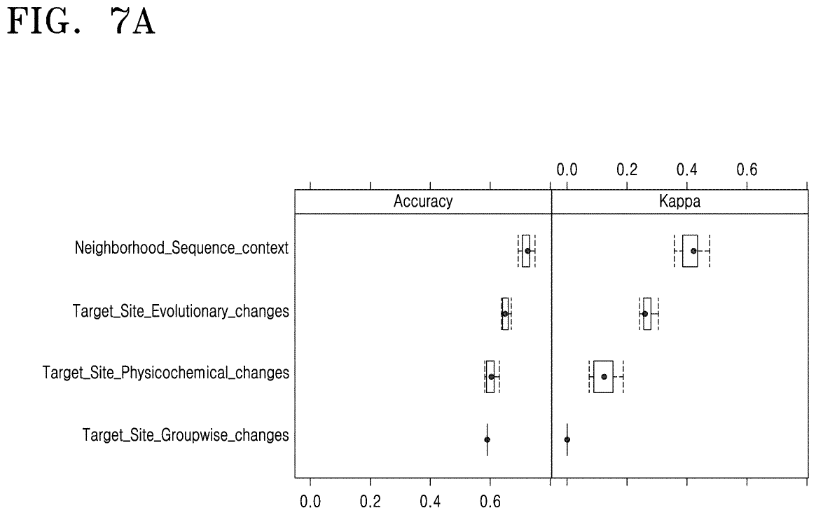

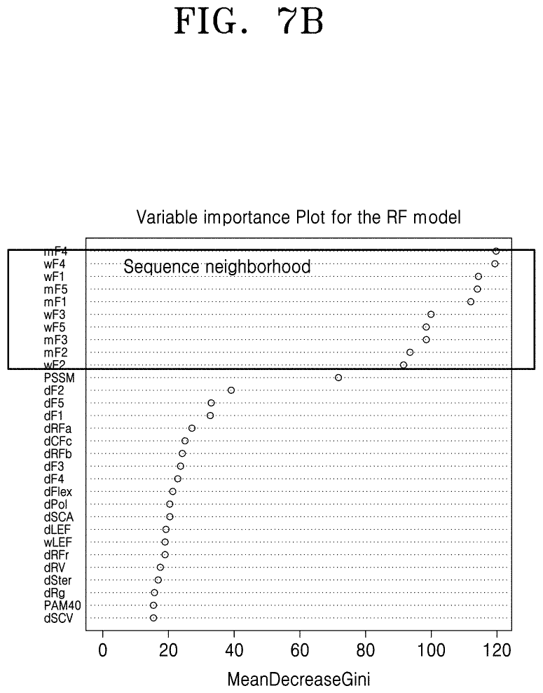

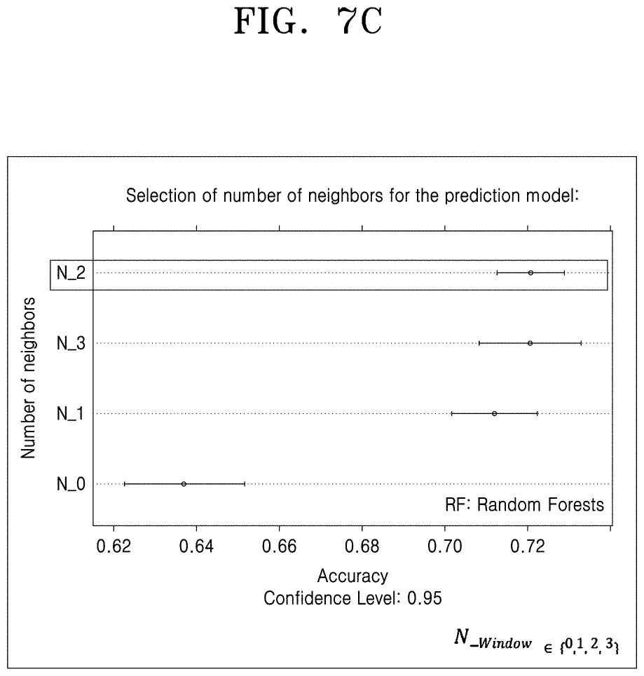

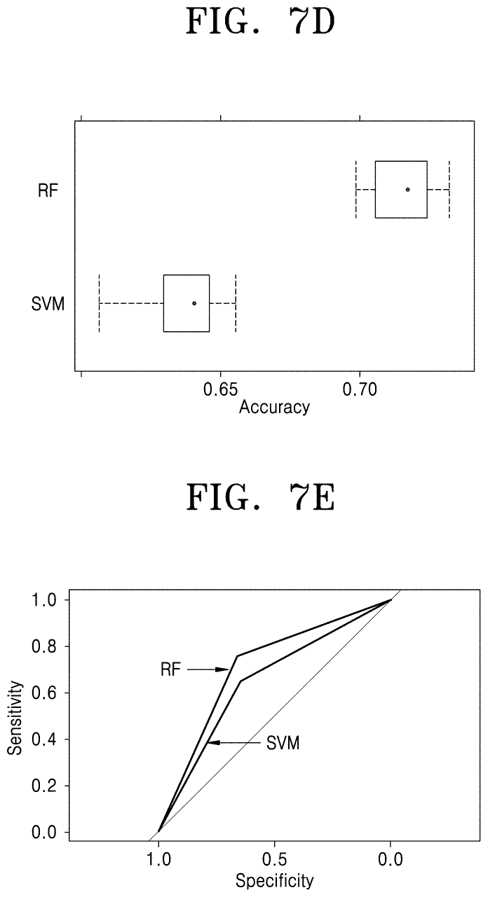

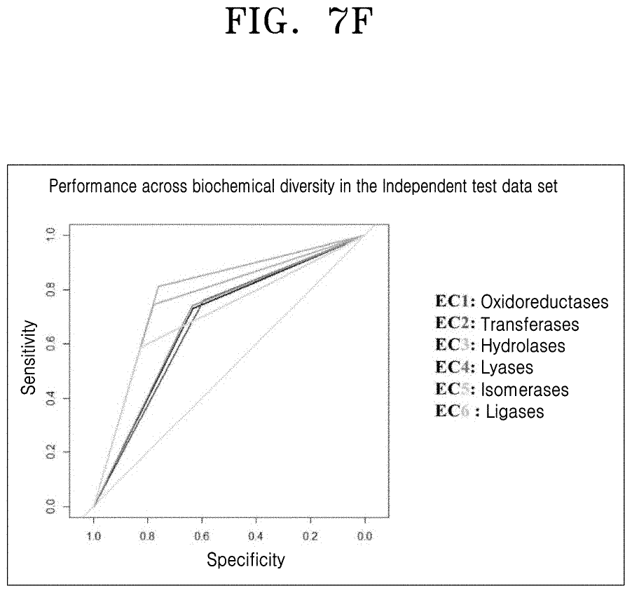

FIG. 7A is a graph illustrating different feature combinations for neighborhood sequence context, target site evolutionary changes, target site physicochemical changes and target site group-wise changes, according to the embodiment as disclosed herein.

FIG. 7B is a graph illustrating the importance of sequence features in the Random Forest (RF) model, according to the embodiment as disclosed herein.

FIG. 7C is a graph illustrating selection of number of neighbors for the prediction model development, according to the embodiment as disclosed. Shown in FIG. 7C, is an exemplary embodiment in which the biological features were extracted, and an optimal number of neighbors are selected for prediction purposes. The analysis showed that when the neighborhood of two residues on either side of the target residue was considered, the best performance is obtained. Although, two and three neighbors performed almost similarly, the model is selected using two neighbors on either side, to enable better prediction with minimal loss, specifically for cases of terminal residues.

FIG. 7D illustrates the performance of the prediction models during the training phase, according to the embodiment as disclosed herein.

FIG. 7E illustrates the performance of the prediction models upon large-scale validation on an independent test set, according to the embodiment as disclosed herein.

FIG. 7F illustrates the performance of the best prediction model across different enzyme classes in the independent test set, according to the embodiment as disclosed herein.

As shown in the FIGS. 7D, 7E and 7F, considered as an example, the best model is tested on a considerably large independent test dataset comprising of 2519 mutations. Though this problem is particularly challenging at a sequence level, the proposed model showed an accuracy of 72% on average, which is very encouraging. It is consistent for various Enzyme Commission (EC) classes demonstrating its ability to handle biochemical diversity.

In an example, for demonstrating the robustness of the proposed method, another set of eight sites is considered for further validation. The method facilitates reduction of the search space while considering candidates for experimental validations. As shown in the table 1, the hatching are favorable outcomes predicted by the proposed method. The proposed model has been successfully able to reduce 85% of the total assessment possibilities, some of which are also experimentally validated as shown in the tick marks.

TABLE 1

| Enzyme | Site | WT | A | C | D | E | F | G | H | I | K | L | M | N | P | Q | R | S | T | V | W | Y | AN |
|---|---|---|---|---|---|---|---|---|---|---|---|---|---|---|---|---|---|---|---|---|---|---|---|
| Malate synthase | 338 | R | | | | | | | | | | | | | | ✓ | — | | | | | | 0 |
| Proline dehydrogenase | 540 | S | ✓ | | | | | | | | | | | | | | | — | | | | | 0 |
| Phosphoribosyltransferase | 150 | D | | | — | | | | | | | | | | | | | | | | | | 6 |
| Thymidylate synthase | 229 | N | | | | | | | | ✓ | | | | — | | | | | | | | | 0 |
| Dihydrofolate reductase | 64 | N | | | | | ✓ | | | | | | | — | | | | ✓ | | | | | 0 |
| | 35 | Q | | | | | | | | | | | | | | — | | ✓ | | | | | 14 |
| Phosphate synthase | 101 | S | ✓ | | | | | | | | | | | | | | | — | | | | | 16 |
| | 97 | T | | | | | | | | ✓ | | | | | | | | | — | | | | 6 |

Amino acids, A-Y, are denoted in one-letter code and enzyme (e.g., Malate synthase, Proline dehydrogenase, Prosphoribosyltransferase, and Thymidylate synthase) are in the ligand binding region.

The embodiments disclosed herein can be implemented using at least one software program running on at least one hardware device and performing network management functions to control the elements.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of exemplary embodiments, those skilled in the art will recognize that the embodiments herein can be practiced with modification within the spirit and scope of the embodiments as described herein.

What is claimed is:

1. An electronic device for predicting a change in a functional property of a target biomolecule, comprising:
a data extraction unit configured to obtain a plurality of biomolecules and mutation data associated with at least one functional property;
a sequence feature detector configured to:
determine a net change in the at least one functional property based on at least one parameter associated with the mutation data for each biomolecule at a site, and
encode a plurality of sequence features for the mutation data associated with each biomolecule;
a prediction model generator configured to configure a prediction model to detect a relationship of the sequence features and the net changes in the at least one functional property of the mutation data associated with each biomolecule;
a functional property change analyzer configured to predict a change in the at least one functional property of a target biomolecule based on the prediction model for the mutation data at a user specified site; and
optionally a communicator configured to communicate internally between internal hardware components and with external devices and/or a user.

2. The electronic device of claim 1, wherein the plurality of biomolecules and the mutation data associated with at least one functional property are obtained by:
obtaining at least one amino acid sequence for each biomolecule;
mapping the mutation data on sequence information for each biomolecule;
mapping a parameter associated with the at least one functional property for the mutation data; and
generating a dataset and obtaining homology information for each biomolecule based on protein domain knowledge.

3. The electronic device of claim 1, wherein the mutation data comprises a substitution site in the biomolecule, a wildtype residue associated with the biomolecule at the substitution site, one or more mutant residues associated with the biomolecule at the substitution site, or a combination thereof.

4. The electronic device of claim 1, wherein determining the net change in the at least one functional property comprises:
obtaining a range of a starting value and an endpoint value for the at least one functional property, with respect to a site and residue, for a ligand and a set of assay parameters;
computing the net change as the difference between the starting point value for the mutant and the starting point value for the wildtype residue at the site; and
determining the net change in the at least one functional property.

5. The electronic device of claim 1, wherein encoding the plurality of the sequence features for the data associated with the biomolecule comprises encoding a combination or sub-combination of properties of the wildtype residue, properties of the mutant residue and property differences upon mutation at the target site and the target site neighborhood, wherein the target site neighborhood comprises the target site and at most three residues on either side of the target site.

6. The electronic device of claim 5, wherein the combination or sub-combination of the properties encoded for the target site and target site neighborhood is obtained by at least one of:
selecting at least one of a set of physicochemical and evolutionary properties for the target site;
selecting genetic and chemical fingerprints of enzyme architecture and biochemical process involving interactions specific to a predefined criteria for the target site and the target site neighborhood; and
selecting factors comprising at least one of a polarity index, a secondary structure factor, a molecular size, a relative amino acid composition and an electrostatic charge for capturing a sequence context.

7. The electronic device of claim 6, wherein the selected set of physicochemical and evolutionary properties for the wildtype and the mutant residue at the target site and the target neighborhood are encoded using a combination of a numeric component and a categorical component.

8. The electronic device of claim 6, wherein the sequence context is encoded as an average of the factors computed over the wildtype and mutant neighborhood of the target site.

9. The electronic device of claim 7, wherein the numeric component of the sequence-features comprises a physicochemical property, wherein the physicochemical property comprises residue flexibility, residue volume, side-chain angle, radius of gyration, side-chain volume, molecular weight, polarity, relative frequencies in alpha-helix, relative frequencies in beta-sheet, relative frequencies in reverse-turn, Chou-Fasman parameter of coil formation, steric parameter, localized electric effect, or a combination thereof.

10. The electronic device of claim 7, wherein the categorical component encodes a wildtype residue to mutant residue change in amino acid physicochemical group using one or more properties, wherein the one or more properties comprises size, aliphaticity, aromaticity, polarity and charge.

11. The electronic device of claim 6, wherein an evolutionary property is determined using historically recorded data and the biomolecule homologs.

12. The electronic device of claim 6, wherein an evolutionary property is indicative of site specific residue information transfer.

13. The electronic device of claim 1, wherein the prediction model is configured by:
grouping the mutation data associated with each biomolecule into two groups based on a pre-defined grouping criteria applied to the net change in the at least one functional property;
partitioning the mutation data associated with each biomolecule into a training dataset and a testing dataset based on a pre-defined partitioning criteria;
presenting sequence features extracted from the biomolecules in the training set for detecting one or more partitioning functions describing the relationship between the sequence features and the net change in the at least one functional property;
selecting a best partitioning function based on a pre-defined assessment criteria; and
assessing classification function performance of the selected partitioning function on the testing dataset.

14. The electronic device of claim 13, wherein the pre-defined grouping criteria for grouping the mutation data associated with the biomolecule is based on one of increase and decrease in the net change of the at least one functional property.

15. The electronic device of claim 1, wherein the at least one functional property comprises affinity of the target biomolecule towards a ligand.

16. The electronic device of claim 1, wherein the target biomolecule comprises an enzyme.

17. The electronic device of claim 1, wherein the prediction model is used to reduce the number of amino acids to be evaluated by mutagenesis of the target biomolecule at the user-specified site for a selected change in the at least one functional property.

18. The electronic device of claim 1, wherein the prediction model is used to identify at least one site in the target biomolecule which provides a selected change in the at least one functional property.

19. A method for predicting a change in a functional property of a target biomolecule using the electronic device of claim 1.
